(19) United States
(12) Patent Application Publication
(10) Pub. No.: US 2010/0152548 A1
(43) Pub. Date: Jun. 17, 2010

(54) MEASUREMENT DEVICE, SYSTEM AND METHOD

(76) Inventor: Salla Koski, Helsinki (FI)

Correspondence Address: YOUNG & THOMPSON, 209 Madison Street, Suite 500, Alexandria, VA, 22314, US

(21) Appl. No.: 12/594,899

(22) PCT Filed: Nov. 20, 2007
(86) PCT No.: PCT/FI07/50624

§ 371 Date: Dec. 2, 2009

(30) Foreign Application Priority Data
Nov. 20, 2006 (FI) FI20065735

(51) Int. Cl. A61B 5/00

(52) U.S. Cl. 600/301

(57) ABSTRACT

A user portable measurement device (100) includes a measurement unit (174) for measuring and monitoring human health information, a user interface (175) for presenting information based on a measurement result, elements for storing information relating to the care of the user (173), and elements for forming activity instructions on the basis of the care information and at least one measurement result. The user interface (175) of the measurement device (100) preferably includes a display for presenting characters and/or symbols and elements for forming a tone and/or speech. The device is especially suitable for e.g. monitoring and care of overweight and diabetes. A system having many user devices and a server for acquisition and storage of the measurement data is also described. The server may further be used to provide an access to game or chat program services to motivate or help individual users of the measurement devices.

### FIELD OF INVENTION

An object of the invention is a measurement device, a system and a method for measuring and monitoring data which relates to human health. The invention is especially applicable for e.g. monitoring and care of overweight and diabetes disease.

### BACKGROUND TECHNOLOGY

A person with a diabetes disease must take especially good care for his/her diet, general condition, regular rest and medication. The care of the diabetes disease also demands regular concentration measurement of blood sugar i.e. blood glucose. The measurement must be performed even several times a day. The measurement commonly takes place by taking a small sample of blood from a finger tip, which sample is let to absorb into a sample test dot of a sample test strip. The glucose concentration is measured from the strip with a portable measurement device, for example. The sample test strip is placed to the measurement device, after which the measurement device performs blood glucose measurement from a blood sample situated at the sample test dot, and shows the measurement result on the digital display of the measurement device. When a person knows the glucose concentration he/she may assess, whether he/she has need for an instant care action. If the measured blood glucose concentration is too high, e.g. more than 15 mmol/l, the care action is generally adding insulin to blood by punching. If the measured blood glucose concentration is too low, e.g. less than 3 mmol/l, the care action is generally taking drink or food which contains carbon hydrate. In addition to assessing an instant care action the blood glucose concentration gives information on requirement of eating, physical exercise, rest or medication. When using the program, the measured data can be input to the computer for an analysis performed by the program.

In addition to measurement devices of blood sugar concentration there are also programs to be installed in a personal computer, with which a person can monitor the development of blood glucose concentration.

In spite of the fact that portable measurement devices and care equipment are available for caring diabetes, the monitoring and care is insufficient for a large portion of people suffering diabetes. This is caused by the following exemplary factors:

Children do not understand or remember the matters relating to diabetes care and they are not interested in them. On the other hand, parents do not have a possibility to provide persistent looking after the care of the diabetes of a child. Therefore, following a care program would also require a child's high motivation and interest in the care.

The care of diabetes is considered difficult and restricting because many equipment needed for the care should always be carried along. It may also be easy to forget keeping the equipment along.

Many users are not able to use the available measurement devices and they can possibly not assess the actions that are required based on the measurement results, or to follow the program and time table related to the care.

The doctor taking care of a person does not always have enough information on realisation of the care and of the development and the variation of the glucose values.

Further, in case of small children the care is made inconvenient by the fact that a personal assistant is required for them at school.

There also exist systems, by which it is possible to transfer measurement results of the measurement devices to user's work station and possibly further to personnel of the health care, and to perform analyses on the information. Such systems give information on the history of the measurement results, but the advantage they give is smaller in solving the above problems of daily care.

Since diabetes is quite a common disease, insufficient care is a significant problem except for the person himself/herself but also for the society. Therefore, the improvement of care and solving the above problems is important.

### SUMMARY OF THE INVENTION

The object of the invention is to provide a new solution for measuring and monitoring information which relates to health and possibly for performing instant care actions, by which solution the above problems can be decreased or avoided.

The object of the invention is achieved with a solution in which a portable measurement device of a user has measurement means for measuring at least one magnitude which relates to human health, a user interface for showing to the user information based on the measurement result, means for storing information relating to care of the user, and means for forming a reminder and/or activity instructions on the basis of at least one measurement result. The user interface of the measurement device preferably includes a display for showing numbers, characters and/or graphical symbols, and means for forming a tone and/or speech.

In one embodiment of the invention a measurement unit meant for children comprises a display which shows the measurement results, reminders and/or activity instructions by means of expressions of an avatar. This way measurement results are illustrative for a child. Alternatively or additionally it is possible to show numeric information on the display of the device. According to one embodiment the measurement device shows the measurement result, reminder, and/or history of the measurement results acquired by the measurement device visually by means of a moving object, which is shown on the display.

Further, the measurement device meant for children can be on a wristband, on a belt or on neck, whereby it is easy to keep the measurement device along. Preferably, a care program utilising user specific data is stored into the measurement device, by which program the measurement device gives reminders and activity instructions according to the care program stored into the measurement device.

In one embodiment according to the invention the measurement device has reception means for receiving information relating to user's movement from another device which measures the movement of the user. The reception means of the measurement device may also receive from another device, such as a pulse meter, data of heart pulse rate, whereby it is possible to measure the intensity of the strain during a physical exercise of a child.

In one embodiment of the invention the system includes a game program which is loaded on a server, for example, to which program the users or a certain user group has an access. It is possible to set into the game program information of each child user's care plan and to transfer measurement results from each user's measurement device to the game program server. This way the child users of the system can play with each other and compete on how well the care plan is realised by each user. Further, in one embodiment of the invention the system includes e.g. a chat program which is loaded on a server, into which program the young users of the system have an access, whereby the users of the system can have contact with each other and discuss matters relating to the disease.

A user portable measurement and monitoring device according to the invention, which comprises a measurement unit for measuring at least one magnitude that relates to human health, and a user interface for presenting to a user information which is based on a measurement result,

is characterised in that

the measurement and monitoring device comprises means for storing data which relates to care of the user,

the measurement and monitoring device comprises means for forming a reminder and/or an activity instruction on the basis of said care data and at least one measurement result, and

the user interface has been arranged to present said reminders and/or activity instructions to the user.

A system according to the invention for measuring at least one magnitude which relates to human health and for monitoring said measurement data, which system comprises several measurement devices, whereby a measurement device is meant for measuring at least one magnitude which relates to health of a certain person and comprises a user interface for performing measurement and possibly for monitoring, for reminders, and for performing care activities, and which system comprises means for acquisition and storage of measurement data from said several measurement devices, is characterised in that the system comprises a measurement device according to the present invention.

A method in which at least one magnitude, which relates to human health, is measured and the measurement result is presented to a user with a user portable measurement device, is characterised in that information relating to care of the user is stored, a reminder and/or an activity instruction is formed on the basis of said care information and at least one measurement result, and said reminders and/or activity instructions are presented to the user.

Preferable embodiments of the invention are described in dependent claims.

In this patent application “measurement device” means a device for measuring a magnitude which relates to human health, such as for measuring blood glucose concentration. However, a measurement device may also include monitoring actions of the measurement results, actions reminding a user, action equipment, sample substrates required in the measurements, etc.

“Measurement unit” means in this patent application a part of a measurement device, which has a measurement sensor and preferably means for defining a value of a measurement result on the basis of a signal received from the measurement sensor. A measurement unit may also comprise a memory and programs for monitoring and reminder functions and for possible other functions of a measurement unit.

“A sample substrate” means in this patent application e.g. a tape or strip, which has sample test dots for absorption and measurement of a blood sample.

In this patent application “a user interface” means broadly means and programs relating to the means, by which a user or his/her performs taking samples, measurements, monitoring, care actions, detects measurement results, instructions or reminders or uses supplementary services. A user interface also comprises e.g. a structure which is at hand in the use and transport of the measurement device.

In this patent application “a child” means a person of e.g. 0-17 years age.

“A user of a measurement device” means in this patent application primarily a person, whose health related values are measured with the measurement device.

“An assistant” means in this patent application a person who assists in performing a measurement and care actions. When a user is a child the assistant may be e.g. his/her parents, school health nurse or a teacher, or when a user is an elderly person an assistant may be e.g. a person of a home help service.

### LIST OF DRAWINGS

In the following the invention is described with help of the enclosed drawings, in which:

FIG. 1 illustrates a block diagram of an exemplary measurement device according to the invention for the measurement and monitoring of health related data;

FIG. 2a illustrates a top view of an exemplary measurement device which is meant for the use of children;

FIG. 2b illustrates examples of symbols to be shown on the display of the device illustrated in FIG. 2a;

FIG. 3 illustrates a block diagram of an exemplary device according to the invention for measuring movement of a user;

FIG. 4a illustrates as a perspective view an exemplary device according to the invention for measuring movement of a user;

FIG. 4b illustrates visualisation of measurement results in an exemplary system according to the invention;

FIG. 5 illustrates a block diagram of an exemplary system according to the invention for measurement and monitoring of health related data;

FIG. 6 illustrates an exemplary measurement device, which is meant for the use of young and adult people, as viewed through the cover; and

FIGS. 7a and 7b illustrate a flow diagram of an exemplary use of a measurement device according to the invention.

### DETAILED DESCRIPTION OF THE INVENTION

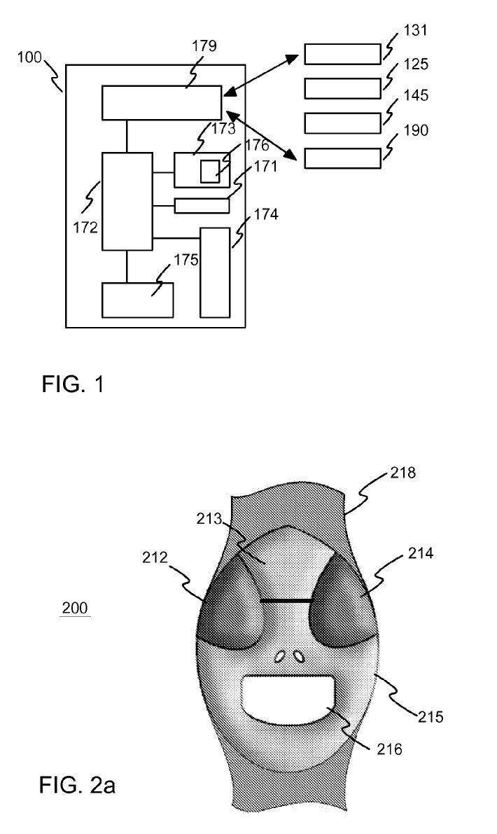

FIG. 1 illustrates a block diagram of an exemplary measurement device according to the invention. The measurement device 100 includes a measurement sensor 174 for measuring a health related magnitude. In the measurement device illustrated in FIG. 1 the measurement sensor 174 measures blood glucose concentration from a blood sample which has been absorbed into a sample test dot. This kind of measurement sensor is preferably based on an optical measurement in a manner, which is prior known as such. Additionally, the measurement device 100 includes preferably one ore several interfaces 179 for receiving data from an external device. An external device may be, for example, a device measuring movement of the user or a device measuring heart pulse, whereby the external device gives correspondingly information of movement and/or heart pulse rate of the user to the measurement device. It is also possible to integrate e.g. a sensor measuring heart pulse rate into the measurement device.

The measurement sensor preferably includes an amplifier, an analogue-digital converter and other required electronics so that the signal received from the sensor can be fed to the input of the processor 172. The processor saves the measurement results into a memory 173 for later use. Also the programs 176 controlling the processor have been stored into the memory 173 of the measurement device. Further, data relating to the care program of the user is stored, which data is preferably user specific.

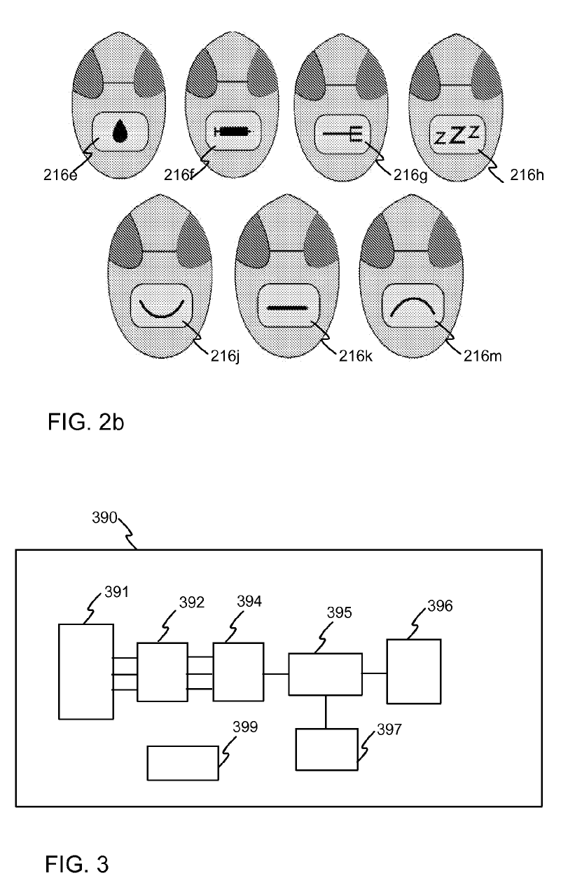

The measurement device also has a display 175, which displays the measurement result and possibly other reminders or activity instructions. In a measurement device meant 100 for children there is preferably a display, which shows the measurement result, reminders and/or other activity instructions as expressions of an avatar. One such measurement device is illustrated in FIG. 2. This measurement device, meant for children, is includes in a wrist band and the top surface of the measurement device form an avatar, such as face of a duck. On display 175 e.g. a mouth area of the face is shown, whereby the mouth position shown on the display gives a desired expression for the avatar. This kind of manner of presentation is illustrative for children. The information to be shown for the user can naturally also be shown on a customary display which shows number and letter characters. The user interface of the measurement device preferably also has means for producing voice. They may produce tones or speech, by means of which the user is guided and given information. The audio signals corresponding to tones or speech can be preferably formed in the processor by means of data, such as a program or parameters, stored in the memory.

The measurement device has data transfer means 171, by means of which it is possible to transfer data between the measurement unit and a terminal equipment of a user. The terminal equipment may be e.g. a computer or a mobile communicator, such as a communicator of UMTS or GSM cellular system. The data trans-fer may take place in a wired manner, e.g. via a USB port (Universal Serial Bus), or in a wireless manner e.g. via a Bluetooth interface. Through the data transfer means it is possible to transfer measurement results which are stored by the measurement device to a terminal equipment and/or to transfer programs or personal measurement and reminder parameters from the terminal equipment to the measurement device.

The data transfer means 171 of the measurement device can alternatively connect directly to a data transfer network, whereby it is possible to transfer data another device which is in connection via the network. Then the data transfer means may be e.g. GSM or UMTS module to which a SIM card of a user may be connected.

The data transfer means may also transfer data between the measurement device and other devices which are part of the system. For example, a measurement device can receive from an insulin injector 131 information that the user has received an insulin dose. The measurement device may also use this information as an acknowledgement for the given activity instruction and as information which affects the reminders and activity instructions according to the care program. The measurement device may also transfer information to the insulin injector on the amount of insulin to be dispensed. Then it is, however, preferable that the amount of insulin is checked by the user/assistant before giving the dose of insulin.

Further it is possible that the data transfer means 171 of the measurement device receive data relating to movement of the user from a separate device which measures movement of the user. This kind of a device measuring movement measures the movement of the user preferably in three orthogonal directions and transmits data of the amount and character of the movement to the measurement device. The measurement device may further use this data relating to the movement for determining reminders and activity instructions which belong to the user's care program.

FIG. 2a illustrates one measurement device 200 according to the invention, which device is meant for children. The measurement device has been designed to be used on user's wrist and it has an attachment for a wrist band 218. Alternatively the device can be kept on neck or waist, as such or in a protective bag. The outer form of the measurement device 200 is an avatar which resembles a head of a duck, and the user interface of the measurement unit forms a part of the avatar. The user interface has a display 216, by means of which the measurement results are presented. The display has been arranged to present the measurement results as expressions of an avatar, for example a good measurement result, which is within predetermined limits, is presented as a glad expression, and a bad measurement result, which is outside predetermined limits, is presented as a sad expression. The expression is easily provided by e.g. showing a suitable figure illustrating position of a mouth according to FIG. 2 on a matrix liquid crystal display or on an OLED (Organic Light-Emitting Diode) display. Additionally, the measurement device may have a small sized loudspeaker (not shown in Figure), by means of which it is possible to give for the user tones describing measurement results or reminding on activities, or instructions as speech.

The user interface further has press switches 212 and 214, by means of which the user can activate the functions of the device and acknowledge reminders given by the device. The push buttons may also function as displays in addition to the display 216. For example, by pressing the press switch one can activate the measurement of blood glucose concentration and by pressing the press switch 214 one can acknowledge a performed activity. If it is desired to store a result of a performed measurement the storing can be activated by pressing both press switches 212 and 214 simultaneously. The push buttons can also be used acknowledging reminders or activity instructions given by the measurement device.

The measurement device 200 has preferably an upper part 213, which is detachable from the base part 215. Thereby a measurement strip which is used in a measurement of blood glucose concentration can be settled into the opening situated at upper edge of the base part, in place of the upper part.

In a measurement device according to FIG. 2a there is preferably stored parameters that belong to the user's care program, on basis of which parameters the device can remind the user of e.g. eating, physical exercise, rest, medication and/or measurement of blood glucose concentration at predetermined points of time. The measurement device may present this kind of reminders with e.g. tones which can be acknowledged by the user by pressing a push button of the measurement device. Additionally, the measurement device can also monitor the realisation of the care program on the basis of blood glucose concentration and movement information received from acceleration sensors of another device. Such data acquired by the measurement device are preferably stored into the memory of the memory of the measurement device, from which they can be read and transferred to a terminal equipment of the user or to a central computer of the nursing staff.

It is easy to carry along the device illustrated in FIG. 2a, which device includes a wrist band. In the measurement device a presentation of measurement results as expressions of an avatar is used, which is a very illustrative form of presentation for children. The results may also be presenter as numbers.

FIG. 2b illustrates some examples of symbols, which can be presented on the display of the measurement device. In the first Figure a drop is shown on the display 216e, which drop means a reminder for measuring blood glucose concentration. In another Figure an insulin injector is shown on the display 216f, which means an activity instruction for injecting insulin. In the third Figure a fork is shown on the display 216g of the measurement device, which means an activity instruction for eating. In the fourth Figure Z characters are shown on the display 216h of the measurement device, which means an activity instruction to sleep. In the fifth Figure smile expression is shown on the display 216j of the measurement device, which means that the result of the blood glucose concentration measurement has been good. In the sixth Figure an expression is shown on the display 216k of the measurement device, which means that the result of the blood glucose concentration measurement has been weak because the sugar concentration of blood has been too low. In the seventh Figure a sad expression is shown on the display 216m of the measurement device, which means that the result of the blood glucose concentration measurement has been poor because the sugar concentration of blood has been too high.

The symbols and patterns described above belong to the most essential symbols which describe measurement results, reminders and activity instructions in the present embodiment. A more thorough selection of symbols and patterns to be shown is described in the following table 1.

TABLE 1
An example of a selection of symbols/characters that can be displayed.

| | |
|---|---|
| [symbol] | Pulse |
| [symbol] | Blood sugar measurement |
| [symbol] | Rest |
| [symbol] | Nutrition |
| [symbol] | Medication |
| [symbol] | Physical exercise |
| [symbol] | Additional function for example, in receiving data from another device |
| [symbol] | Fanfare for good following of care program |
| [symbol] | Reminder to send measured data to the care team/contact to the care team |
| [symbol] | Emptying the memory |
| [symbol] | Poor = too low blood sugar > requires actions |
| [symbol] | Moderate = limit value > requires a further measurement after a moment |
| [symbol] | Excellent = desired measurement result > no actions |
| [symbol] | Moderate = limit value > requires a further measurement after a moment |
| [symbol] | Poor = too high blood sugar > requires actions |

Table 1 includes symbols which present action instructions, such as pulse measurement, measurement of blood sugar concentration, rest, eating, medication, physical exercise data transfer from the device and resetting the memory. There are also two symbols which mean “moderate measurement result” belonging to the symbols presenting measurement results. In such situation the measurement result is within the range between good and weak or correspondingly within the range between good and poor. This kind of measurement result means at the same time an activity instruction to perform the measurement again after a predetermined period of time.

One must note that also other groups of people than children can use a device according to FIGS. 2a and 2b, used on a wrist band. Such user groups are adults and elderly people, for example. In such a case the housing of the measurement device can be preferably designed pleasing for the concerned user group concerning the form of the housing as well as the user interface of the device. It must be noted that also within a child group there may be various user groups for which own versions of the device can be designed. Concerning age, such groups may be babies and teen-agers. The device can also be used to other purposes than caring diabetes, such as for caring and monitoring overweight. One can monitor and guide eating, rest, keeping general condition and medication of the user with the device. Avoiding and care of overweight is naturally also prevention of diabetes disease.

FIG. 3 illustrates a block diagram of an exemplary device 390 according to the invention for measuring movement of the user. The device 390 has movement or acceleration sensors 391, the amount of which is preferably three. The movement sensors have been placed orthogonally related to each other for measuring movements in perpendicular directions. The signals received from the sensors are amplified in an amplifier 392 and converted into digital form with an A/D converter 394. The digital signals describing movement/acceleration are led to an input/inputs of a processor 395, from which the processor transfers the data into a memory 397 of the device. The processor may also calculate desired characteristic values from signals or filter the signals. Said A/D converter and amplifier may also be included in the processor.

At desired points of time the device can transfer the acquired data to a measurement device according to the invention, which preferably uses the measurement results of movement for determining data to be given for the user, such as reminders or activity instructions. The device may also alternatively or additionally transfer the data directly to a computer or to another monitoring system. The transfer of the data takes place by means of a transmission part 196 wirelessly with e.g. radio data transfer of Bluetooth type. Alternatively the data may be transferred with wired connection such as e.g. USB interface. The device of FIG. 3 also includes a energy source 199, such as a rechargeable or disposable battery. The device may also include user interface means such as push buttons, display and sound forming means for forming tones and/or speech. The device may also be independent device which is usable without the present measurement device. Even in such a case the device has preferably an interface to some device, such as a computer or a mobile communicator, which receives/processes the measurement results.

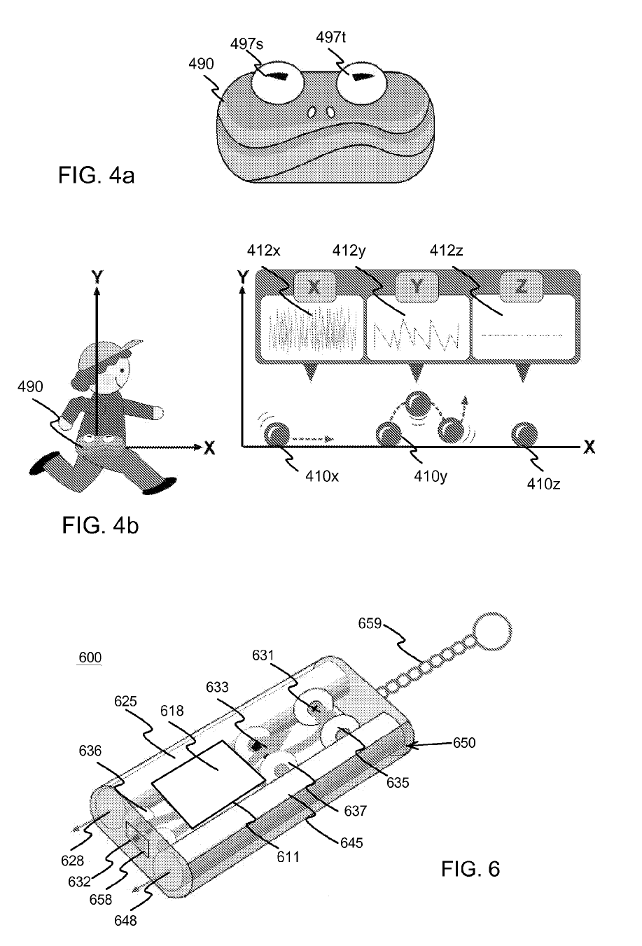

FIG. 4a illustrates an exemplary device 490 according to the invention for measuring movement of a user. The device has two push buttons 497a and 497t, which can function as e.g. on/off buttons. If required, the push buttons may also function as displays. A device measuring movement is preferably attached to the user, for example, to a belt of the user. The device can be alternatively placed into a burn bag, but in such a case the burn bag must preferably be of a suitable size so that the device is held in the same orientation in relation to the user. The device must be kept in the same orientation in relation to the user so that the signals from the sensors of the device describe desired directions of movement.

The measurement results of devices which are illustrated in FIGS. 3 and 4a can be preferably presented on a display in a visualised way of presentation. This display may be included in a measurement device according to the invention, a computer, a mobile communicator or similar, into which the movement measurement data has been transferred. Also the device itself which measures the movement may include a display with which the measurement results can be presented.

FIG. 4b illustrates one such visualised way of presentation. Signals are received from acceleration sensors of the device 490, which measures movement, which signals, after possible filtering, are illustrated on time axis at the upper part of FIG. 4b. The graph 412x illustrates momentary values of acceleration of the user in forward direction as a function of time, the graph 412y correspondingly illustrates vertical acceleration values and the graph 412z illustrates values of horizontal acceleration. It must be noted that instead of acceleration it is also possible to examine movement speeds i.e. integral of the acceleration. In the lower part of FIG. 4b the movement is presented in a visualised manner as bouncing, rolling or stationary. When a user is running directly forward a rolling ball is achieved as a visualised presentation signal 412x, a ball bouncing up and down as a presentation for signal 412y, and a stationary ball as a presentation for signal 412z. By combining these components, a forward bouncing ball can be presented to the user. Instead of a ball the movement can be visualised by using other alternative objects, such as animals or cartoon characters. In a corresponding visualised manner it is possible also to present measurement results, reminders or activity instructions in a measurement device according to the present invention. Also, data acquired by the measurement device can be examined by means of a visualised presentation either on the display of the measurement device or on another device into which the data stored by the measurement device has been transferred. The conversion of a measurement value of a certain magnitude into visualised presentation takes place most preferably with a processor of the presenting device, wherein there is a conversion program controlling the processor, stored in a memory which is used by the processor.

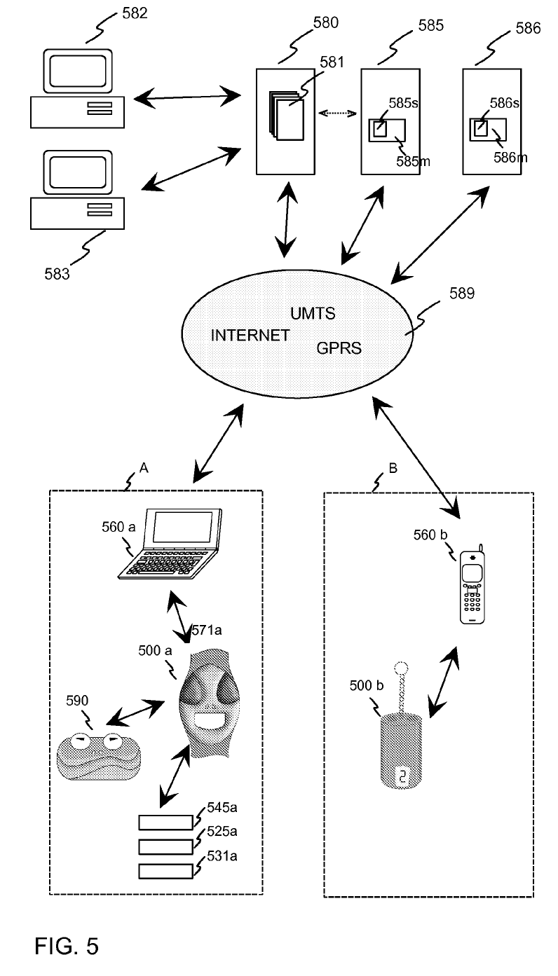

FIG. 5 illustrates an exemplary measurement and monitoring system according to the invention. The system includes several measurement devices, of which measurement devices 500a and 500b are shown in the Figure. The measurement devices are e.g. wrist band measurement devices 500a of child users or measurement devices 500b of young/adult users.

The devices A of a child user include in addition to a wrist band type measurement device sample test strips 531a, sample taking prick 525a, and an insulin injector 545a. The devices may include data transfer means, with which it is possible to get information to the measurement device on the matter that the user has been given insulin. The measurement device can use this information in determining measurement and other activity instructions. The devices of user A also include a device 590 which measures movement preferably in three orthogonal directions, and transfers data relating to the movement to the measurement device 500a. Said data transfer takes place preferably in a wireless manner, with a Bluetooth type radio data transfer, for example.

Additionally the user typically has a personal data processing means, such as a laptop computer 560a, mobile communicator 56+b or a corresponding device into which the measurement results and the monitoring results of the measurement device are transferred. The data processing means is further in connection with a central computer 580 and/or servers 585 and 586 via a data transfer network 589. Additionally, the system includes terminal equipment 582, 583 of the nursing staff, which terminal equipment is connected to the central computer. The servers 585 and 586 may also provide supplementary services for the users.

The central unit 580 comprises a database 581, into which personal information and information relating to the disease of the measurement device users is stored. The central unit can be e.g. a central computer of regional health care. The central unit 580 can also be a computer unit or a server which is separate from other database of the health care, and which comprises only self care information relating to a certain disease, such as information on blood glucose concentration measurement and care monitoring. Nursing staff, such as a nurse and doctor, has access to the information of the central unit. The access may take place e.g. with terminal equipment 582 and 583 after logging in the system.

The measurement devices have data transfer means, by means of which data can be transferred between the measurement device and a terminal equipment of a user, such as a personal computer, The data transfer may take place in a wired manner, e.g. via an USB port (Universal Serial Bus) of the terminal equipment, or in a wireless manner e.g. via a Bluetooth interface. Through the data transfer means it is possible to transfer measurement results stored in the measurement device to terminal equipment and/or to transfer programs of the measurement device or personal measurement or reminder parameters from the terminal equipment to the measurement device.

The data transfer network 589 can be e.g. a wired Internet or a wireless UMTS or GPRS data transfer network. It must be noted that the data transfer means of the measurement device can alternatively connect directly to the data transfer network 589 in such a way that e.g. the transfer of the measurement data to the central device 580 can take place without a terminal equipment 560a, 560b, and the central device may update the programs or parameters of the measurement device directly without terminal equipment. In such a case the data transfer means can be e.g. a GSM or UMTS module, into which a SIM card of the user may be connected.

In the system illustrated by FIG. 5 the user interfaces of the measurement devices of groups may differ from each other in regards to action means i.e. sample taking prick and insulin injector. When a measurement of blood glucose concentration is performed to a child, assistance is needed from an assistant, such as a parent or a health nurse. Thereby the assistant takes care of the concerned means, and thus it is preferable to have the blood sample prick 525a and the insulin injector 545a separate from the measurement device 500a which is kept on the child's wrist. Also an elderly person may need an assistant for performing a measurement and a care action, whereby the assistant preferably has the action means, separate from the measurement device.

In the system illustrated in FIG. 5 the action means may be integrated into measurement devices in such measurement devices which are meant for young and adult people. This way the action means are easy to carry along with a user and they are always available when required. The sample prick and the insulin injector can be integrated into the measurement device in such a way that they can be used without detaching them from the measurement device. The measurement device may also include the sample test dots or strips. This kind of a measurement device is illustrated later in FIG. 6. Alternatively, said action means may also be thus attached to the measurement device that they can be removed from the measurement device for use e.g. after removing the cover of the measurement device.

The system according to FIG. 5 also includes a server 585, in which e.g. child users of the system can play with each other. The program stored in the server 585 functions in such a way, for example, that the target information and measurement results of the users are stored and it is possible to compete among the users about how good measurement results are in relation to own target values of each user. The program may calculate by means of targets and measurement results performance points that are shown in the game, whereby the real target or measurement values are not available for other users. The program includes preferably an identification functionality, by which the users log in to use the game.

The users can appear in the game with their own name or with a pseudonym, for example. The right of use of the game can be preferably arranged in such a way that a such user can get a user right to the game who is provided with a measurement devices and/or software of a terminal equipment of a certain user group.

Since child users in the system of FIG. 5 have a measurement device 500a and software of the terminal equipment, it is possible to determine as a condition for a user right that the user must have this type of a measurement device and/or software.

In a corresponding way there may be another game program installed in the game server, which is meant for young users and which has thus functions and a user interface which are designed for young people.

The game server is especially meant for child users, whereby also the software of the measurement devices and terminal equipment of the child user group preferably support the game. Playing the game takes place with the terminal equipment 560a by transferring game related data between the terminal equipment and the server 585 via the data transfer network 590. The measurement results processed in the game are preferably transferred from the measurement device 500a by means of data transfer means to the terminal equipment 560a, from which they are further transferred to the server 585. Alternatively, if the measurement results have been transferred to the central device in a corresponding manner, they can be transferred to the game server 585 from the central device 580 if the user has given a permission for it.

A purpose of a game server is to make the activities relating to diabetes more interesting for a child user. Based on the results achieved in the game honour mentions and prizes are preferably given based on the performance of the users. Also the nursing staff may take part in the guiding of the games and awarding the users.

The system according to FIG. 5 also includes a server 586, which has software maintaining chat sites, where the users can have communication with each other about e.g. matters relating to the disease. Also this program preferably includes a identification function, by which the users log in to the chat sites. The identification may take place e.g. by means of a user code and a password. Also, the user right for a chat site can preferably be arranged in such a way that a user provided with measurement device or a program of a certain user group can have a right of use to the chat site. Separate chat sites can be established for user groups of different ages. Using the chat site takes place by terminal equipment 560b by forming a connection between the terminal equipment and the server 586 via the data trans-fer network 590 and by performing the identification of the user.

The purpose of the chat server is to assist communication of such people who have the same disease, such as diabetes. This way these people can exchange their experiences and support each other. This is especially important for young people who suffer from diabetes in order to advance the care of the disease.

FIG. 6 illustrates en exemplary measurement device 600 according to the invention, which is meant especially for young and adult users and in which e.g. a sample prick and an insulin injector has been integrated. FIG. 6 illustrates the device without a front cover in order to illustrate the inner parts of the device. The measurement device 600 comprises a measurement unit 611, which has a display 618 at the front surface of the measurement device. The measurement unit 611 preferably comprises a measurement sensor, a processor, and a memory for performing a measurement. The current source, such as batteries, may reside in the measurement unit or separate from it in the device housing 650.

A program stored in the measurement unit includes preferably corresponding measurement and monitoring functions and functions relating to the care program of the user, which were described above in connection with the description of the measurement device meant for the use of children. The measurement unit also has data transfer means for transferring data between the measurement device and a data processing device such as a computer or a mobile communicator. The measurement device may also have data transfer means for receiving data concerning user's movement from a device which measures the movement of the user. Alternatively it is possible to integrate movement sensors and related circuits into the measurement device for measuring and monitoring movement of the user. In such a case the measurement unit must preferably be attached to the user in such a way that the orientation of the measurement device does not substantially change.

The measurement unit illustrated in FIG. 6 also includes a sample taking prick 625 for making a prick into the user's skin. When the sample taking prick is activated the needle 628 of the sample taking prick performs a quick movement back and forth in the direction of the arrow shown in the Figure. The movement is achieved by e.g. setting a spring, which is included in the sample taking prick and connected to the needle, into a tension state and by releasing it when a press switch is pressed.

The measurement device of FIG. 6 also includes an insulin injector 645. When an insulin injector is activated with e.g. a press switch of the device a needle 648 of the insulin injector moves to outside the device bottom as shown by an arrow in the Figure. By activating the push button of the insulin injector again the injector dispenses a required amount of insulin from an insulin container of the injector through a needle 648. The feeding of the insulin preferably takes palace by means of an electrically operating pump of the insulin injector, but the dispensing can alternatively be performed also with a mechanical, manually used dispensing pump.

The measurement device according to FIG. 6 has, instead of a sample test strip, a sample test tape, which has been installed into the device as a roll 631. The roll 631 has been installed to the measurement device around a control shaft 635 in such a way that the roll is able to rotate, and thus it is possible to take tape into use from the roll by pulling the tape. The second end of the tape is led to an opening 658 located at the bottom end of the measurement device. From here the tape is led around the control shaft 636 to the measurement unit 611 and further to the control shaft 633. The control shafts include electrically operating motors, by means of which the tape can be moved on said track. In order to move the tape the tape preferably moves between the control shaft and a freely rotating elastic roll (not shown in the Figure), whereby the tape gets slightly squeezed between the control shaft and the roll.

The sample test tape has at suitable intervals sample test dots, which have chemicals that are required for the measurement. When the measurement is started the processor of the measurement device controls the movement of the sample test tape in such a way that the sample test dot 632 is settled at the opening 658 of the device housing 650. When the blood sample has been absorbed to the sample test dot of the tape, the processor of the measurement device controls the movement of the tape further in such a way that the concerned sample comes to the location of the measurement sensor. After this the measurement unit performs a measurement of the sample, such as glucose concentration measurement of a blood sample. After the measurement is finished, the measurement result is shown on the display 618 of the measurement device, and the processor controls the movement of the sample test tape further so that the measured blood sample comes, for example, out from an opening of the measurement device, whereby the used part of the tape can be cut and disposed. The concerned opening (not shown in the Figure) of the device cover can locate, for example, on the front or back cover of the device. Alternatively, the used tape may remain into a space which is arranged inside the device, until the tape is exchanged to a new one.

As was mentioned above, the movement of the sample test tape preferably takes place by means of electric motors, which are connected to the control shafts and which are controlled by e.g. a processor of the measurement device. It is essential that a sample test dot of the sample test tape can be placed into an accurately correct location at the opening 658 and to the location of the measurement sensor of the measurement device. In order to achieve an accurate location the measurement device preferably has a positioning sensor, which detects details of the sample test tape. When the processor gets information from the positioning sensor that the detail of the tape is located at the sensor, the processor can use this information in the control of the sample test tape. The positioning sensor may detect the location of the sample test dot itself or marks which are arranged to the tape for the positioning. Such marks can be e.g. transverse lines, holes or grooves at the tape. The positioning sensor can most preferably be an optical sensor which is connected to the processor and which detects a mark or a sample test dot when it is located at the sensor position and which then gives e.g. a signal impulse for the processor.

The sample test tape can be located in the device in many alternative ways. The roll of the sample test tape may be e.g. in a perpendicular position in relation to position shown in the Figure so that the movement track of the tape is parallel with the front surface level of the measurement device, and the lateral direction of the tape is perpendicular to the front surface level of the measurement device. Changing the sample test tape can take place in e.g. such a way that the back cover of the device is opened, whereby a new sample test roll can be changed into the place of the used sample test roll, and in connection of the changing the end of the tape is led to the movement track of the tape. It is also possible that the sample test roll and the movement track of the tape has been arranged into a changeable sample test cassette. Such a cassette may include the control shafts and/or the elastic rolls coming towards the control shafts. This kind of a sample test cassette can be changed into the measurement device without touching the sample test tape. The sample test cassette may also include another roll, into which the used part of the sample test tape is collected. Additionally, the sample test cassette may function as a back part and surface of the device cover.

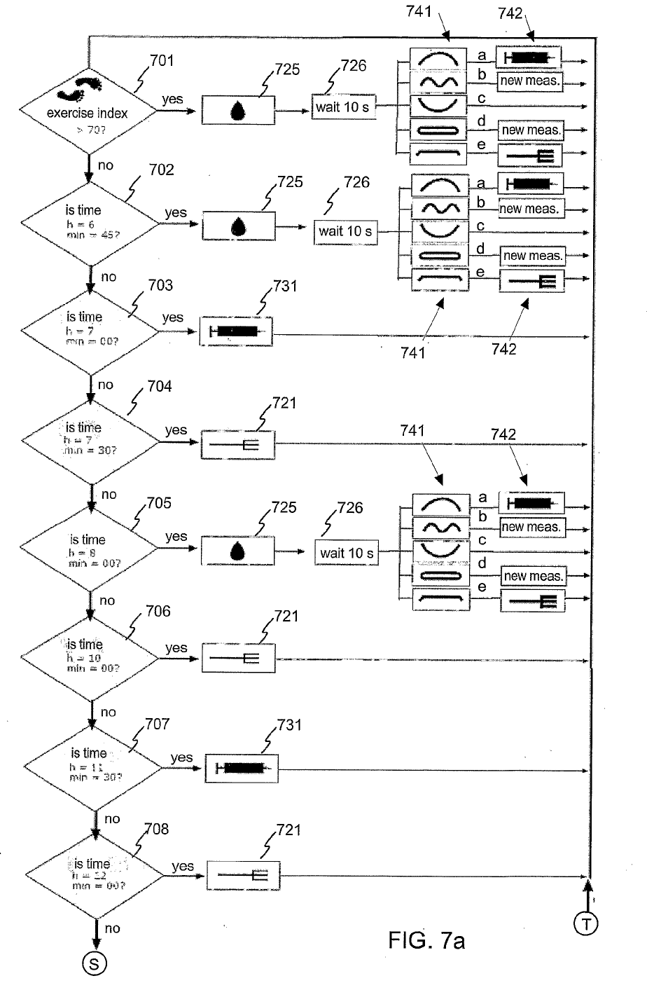

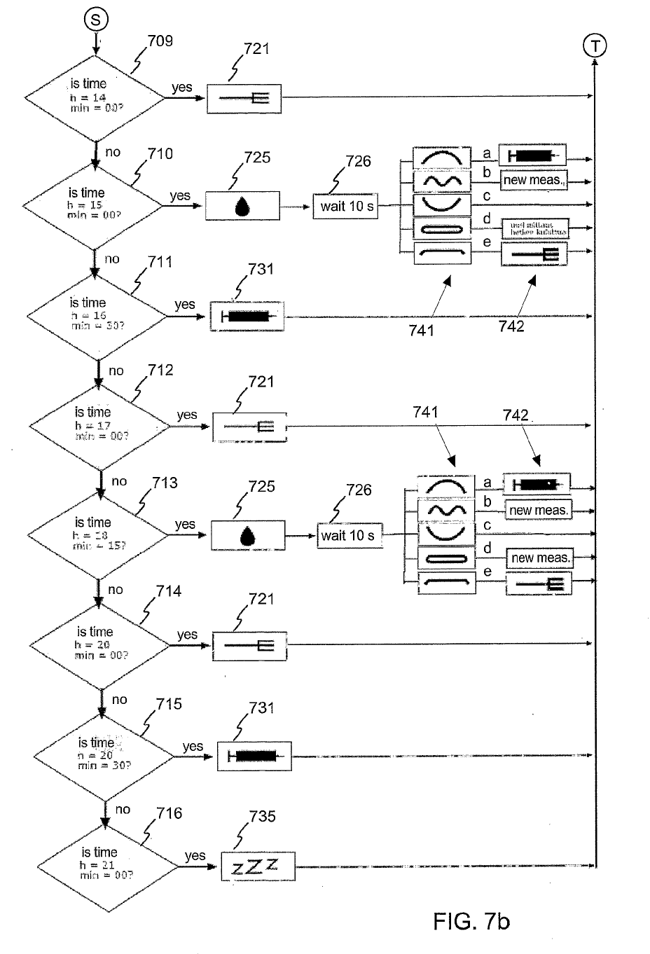

FIGS. 7a and 7b illustrate as a flow diagram one example of using a system and/or measurement device according to the invention. In the method according to the flow diagram, the measurement device performs the checkings according to the phases 701-716 repeatedly. In phase 701 it is checked, whether the movement index of the user is higher than a predetermined limit value, such as 70. The value of the movement index has been transferred to the measurement device from e.g. an external device which measures the movement of the user according to the description of FIG. 1. If the movement index exceeds the threshold value the measurement device gives an activity instruction for the user to measure sugar concentration of blood, 725. The activity instruction can be given on a display, for example, in a form of a symbol or text or in the form of tone or speech. When a sample strip has been set to the measurement device it is possibly necessary to wait for the result given by the measurement device to settle into a right value, phase 726. The length of the required waiting may be e.g. 10 s. After this, the measurement device shows the result on the display of the measurement unit or as a sound, phase 741.

If the result of the measurement is poor, i.e. the sugar concentration of blood is too high, the measurement device gives in phase 742 an activity instruction to give an insulin dose for the user according to the alternative a shown in the Figure. If the measurement result is weak i.e. the sugar concentration of blood is too low, an activity instruction is given in phase 742 to eat, according to the alternative e shown in the Figure. If the measurement gives a limit value result the measurement device gives an activity instruction to perform the measurement again after a predetermined period of time according to alternatives b and d. If the measurement result is good, the result is shown to the user, but in such a case no specific activities are necessary, alternative c. After a possible activity relating to the measurement the user acknowledges the activity performed.

In a method illustrated in FIGS. 7a and 7b the measurement device gives at predetermined points of time a reminder for measuring blood sugar concentration. Such points of time in the described example are at 6:45 o′clock, which reminder is given in phase 702, at 8.00 o'clock in phase 705, at 15:00 in phase 710 and at 18:15 in phase 713. When the blood sugar concentration has been measured corresponding phases 726, 741 and 742 are performed as were described above. After a possible activity relating to the measurement the user acknowledges the activity performed.

Additionally, the measurement device has been programmed to remind giving insulin 731 to the user at predetermined points of time. In the method illustrated in the flow diagram this reminder is given at 7:00 in phase 703, at 11:30 in phase 707, et 16:30 in phase 711 and at 20:30 in phase 715. After the reminder an insuin dose is given to the user, and the user acknowledges the activity performed.

Further, the measurement device has been programmed to remind about eating 721 at predetermined points of time. In the method illustrated in the flow diagram this reminder is given at 7:30 in phase 704, at 10:00 in phase 706, at 12:00 in phase 708, at 14:00 in phase 709, at 17:00 in phase 712, and at 20:00 in phase 714. After eating the user acknowledges the reminder.

Additionally the measurement device reminds the user of rest, 736, at predetermined points of time. In the method illustrated in the flow diagram this reminder is takes place at 21:00, phase 716.

Above some systems and devices according to the invention have been described. The functionality according to the invention is achieved with, in addition to the devices mentioned herein, by storing the programs which relate to the inventive functions and which control the processor/processors into the memories of the devices of the system. Programming a server, terminal equipment personal computers and measurement devices is known as such for a person skilled in the art, and he/she can implement the functions of the present invention on the basis of the description given here.

It must be noted that above only some embodiments of the solution according to the invention have been described. The principle of the invention can naturally be modified within the scope of protection determined by the patent claims, e.g. in details of implementation and areas of use.

The present invention is also not restricted to care of diabetes and monitoring of blood glucose concentration, but the invention can also be used for monitoring other diseases or for monitoring health. As an example, monitoring blood haemoglobin of athletes or people suffering from anaemia can be mentioned.

The present invention is neither restricted to use of displays, push buttons, data transfer types or functions which were described in the above embodiments, but a person skilled in the art can design several alternative implementations in the frame of the inventive features.

1-24. (canceled)

25. A user portable measurement and monitoring device, which comprises a measurement unit for measuring at least one magnitude that relates to human health, and a user interface for presenting to a user information which is based on a measurement result, the user interface comprising at least one display and/or means for forming sound,
characterised in that
The measurement and monitoring device comprises means for storing data which relates to care of the user,
the measurement and monitoring device comprises means for forming a reminder and/or an activity instruction on the basis of said care data and at least one measurement result, and
the user interface has been arranged to present said reminders and/or activity instructions to the user.

26. A measurement device according to claim 25, characterised in that one of the at least one magnitude to be measured is at least one magnitude is blood glucose concentration, heart pulse rate, amount of sleep and/or amount of movement of the user.

27. A measurement device according to claim 25, characterised in that at least a part of said data which is related to the care is user specific.

28. A measurement device according to claim 25, characterised in that the measurement device has been arranged to receive information from an insulin injector about giving insulin to the user.

29. A measurement device according to claim 25, characterised in that it is arranged to receive data about movement of the user from a device which measures movement and/or acceleration of the user.

30. A measurement device according to claim 29, characterised in that information given by a device which measures movement and/or acceleration is arranged to be presented in a visualised form, such as movement of an object presented on a display.

31. A measurement device according to claim 25, characterised in that a reminder and/or an activity instruction to be given for the user has arranged to be dependent on a measurement result which is received from the other device.

32. A measurement device according to claim 25, characterised in that the measurement device has been arranged to present a measurement result, reminder, and/or an activity instruction in connection with an avatar.

33. A measurement device according to claim 25, characterised in that the measurement device comprises means for attaching a the measurement device to the wrist, neck or waist of the user.

34. A system for measuring at least one magnitude which relates to human health and for monitoring said measurement data, which system comprises several measurement devices, whereby a measurement device is meant for measuring at least one magnitude which relates to health of a certain person and comprises a user interface for performing measurement and possibly for monitoring, for reminders, and for performing care activities, and which system comprises means for acquisition and storage of measurement data from said several measurement devices, characterised in that the system comprises a measurement device according to claim 25.

35. A system according to claim 34, characterised in that the system comprises at least one device for measuring the amount and/or quality of exercise of the user, whereby the system comprises means for acquisition and storage of the measured exercise information.

36. A system according to claim 34, characterised in that the system comprises means for transferring data between the measurement device and a central device and/or between the measurement device and a terminal equipment via a data transfer network.

37. A system according to claim 34, characterised in that it comprises a game server and means for storing information relating to a care program and measurement results, means for arranging access for a user of the system into the game program and an arrangement for using said information in the game program.

38. A method in which at least one magnitude, which relates to human health, is measured and the measurement result is presented to a user with a user portable measurement device, characterised in that
information relating to care of the user is stored,
a reminder and/or an activity instruction is formed on the basis of said care information and at least one measurement result, and
said reminders and/or activity instructions are presented to the user.

39. A method according to claim 38, characterised in that one of the at least one magnitude to be measured is at least one magnitude is blood glucose concentration, heart pulse rate, amount of sleep and/or amount of movement of the user.
